(19) United States
(12) Patent Application Publication
(10) Pub. No.: US 2021/0307724 A1
(43) Pub. Date: Oct. 7, 2021

(54) BIOCOMPATIBLE ULTRASONIC COUPLING AGENT FOR ENDOSCOPE AND USE THEREOF

(71) Applicant: ENDOCLOT PLUS CO., LTD, Jiangsu (CN)

(72) Inventors: Cheng Xing, Jiangsu (CN); Xin Ji, Jiangsu (CN); Heng Zhang, Jiangsu (CN); Huihui Xie, Jiangsu (CN)

(21) Appl. No.: 17/263,052

(22) PCT Filed: Jul. 23, 2019
(86) PCT No.: PCT/CN2019/097177

§ 371 Date: Jan. 25, 2021

(30) Foreign Application Priority Data
Jul. 25, 2018 (CN) 201810824196.4

(51) Int. Cl. A61B 8/00; A61B 8/12; A61K 49/22

(52) U.S. Cl. CPC A61B 8/4281; A61B 1/015; A61K 49/22; A61B 8/12

(57) ABSTRACT

Disclosed herein is a biocompatible ultrasonic coupling agent for endoscopes, comprising a biocompatible modified starch and a pharmaceutically acceptable carrier, or comprising an ingredient selected from the group consisting of cellulose, polyvinylpyrrolidone, polyoxyethylene, sodium alginate, glucan, hyaluronic acid, chitosan, light sensitive glue, ultrasonic sensitive glue, pH sensitive glue, gelatin and carbomer, and a pharmaceutically acceptable carrier; wherein the ultrasonic coupling agent produces an acoustic characteristic impedance matching the acoustic characteristic impedance of the human tissues during use for endoscopic ultrasound examination. Disclosed herein is also a kit for endoscopic ultrasound examination, comprising the said biocompatible ultrasonic coupling agent.

### CROSS REFERENCE TO RELATED APPLICATIONS

This application claims priority to Chinese Patent Application Serial No. 201810824196.4, filed on Jul. 25, 2018, entitled “BIOCOMPATIBLE ULTRASONIC COUPLING AGENT FOR ENDOSCOPES AND THE USE THEREOF”, the entire disclosures of which are herein incorporated by reference.

### FIELD OF THE INVENTION

The present invention relates to a biocompatible ultrasonic coupling agent for endoscopic ultrasound examination and the use thereof, as well as a kit for administering the same.

### BACKGROUND OF THE INVENTION

Endoscopic ultrasound (EUS) is an examination technology to assess human cavities, which combines an endoscope and ultrasound with a miniature high-frequency ultrasound probe installed on the top of the endoscope. When the endoscope is inserted into the body cavities, the lesions existing in the cavity mucosa and tissues can be directly observed through the endoscope, and real-time ultrasonic scanning can be performed by virtue of the endoscope to obtain histological characteristics of the hierarchical structure of the cavities and the ultrasound images of the surrounding organs. This combination of the endoscope and ultrasound is beneficial to diagnosis.

In 1980, it was firstly reported in the United States that the examination method combining ultrasound and common endoscope had achieved success in animal experiments, which pioneered the clinical application of endoscopic ultrasound technology. Over more than 20 years of clinical practice, endoscopic ultrasound technology has become more and more mature, with increasingly extensive application. So far, endoscopic ultrasound technology has widely been used in examination of digestive tract, uterus and vagina, bladder and ureter, and bronchus and the like.

In recent years, endoscopic ultrasound technology has also been gradually applied to endoscopic minimally invasive surgeries, such as detection of surrounding organs in laparoscopic and thoracoscopic surgeries. In addition, fine needle aspiration biopsy has also significantly improved lesion confirmation rate by virtue of endoscopic ultrasound technology. Currently, interventional diagnosis and treatment by using endoscopic ultrasound technology are one of the worldwide hot spots of endoscopic technology.

The endoscopic ultrasound technology can be used:
i) to determine the origin and nature of human submucosal tumors, evaluate the stage of the tumors before surgery, measure the depth and extent of tumors' invasion, and identify whether the tumors are benign or malignant,
ii) to determine the depth of tumors' invasion in cavities and the possibility of surgical resection,
iii) to detect lesions of surrounding organs and to perform differential diagnosis; and
iv) to determine treatment efficacy.

Theoretically, the endoscopic ultrasound technology relates to application of medical ultrasound to the specific part of the human body, i.e. within human cavities. The ultrasonic coupling agent is used to fill the space between the ultrasound probe and the surface of the tissues, with which the probe contacts, so as to eliminate the influence on ultrasound penetration due to the air in the said space. Furthermore, the ultrasonic coupling agent plays transitional role to reduce the acoustic impedance difference between the probe and the tissue, thereby reducing the reflection loss of ultrasonic energy at the interface between the ultrasound probe and the surface of the tissues, with which the probe contacts. However, the traditional ultrasonic coupling agents such as Bok-DP (trichlorohydroxydiphenyl ether), benzalkonium chloride, benzalkonium bromide and triethanolamine cannot be applied to human body due to their toxicity, poor biocompatibility and absorbability, and cannot be adhered to the sites to be detected and treated due to their poor bioadhesion. And no device is available to deliver the viscous ultrasonic coupling agent through the narrow lumen of the endoscope to the site to be detected. Due to the particularity of ultrasound endoscopes for human cavities and the limitations of the traditional ultrasonic coupling agents, there is no dedicated ultrasonic coupling agent for endoscopic ultrasound examination which meets clinical needs. Currently, water is used as a coupling media for the endoscopic ultrasound examination in the digestive tract. However, the use of water as a coupling agent in the endoscopic ultrasound examination leads to the following issues:
i) loss of ultrasonic energy, reduction of resolution and generation of obscure images,
ii) inability to unfold the wrinkles of the natural cavities of the human body, which reduces the effect of ultrasound detection,
iii) high fluidity, unable to retain the water in the specific sites to be examined.

Clinically, it needs to inject 500 ml-1000 ml of water for upper gastrointestinal ultrasound examination, even up to several thousand millilitres, which greatly reduces the comfort of the patient and increases the workload of the medical staffs, and
iv) accidental inhalation of water into the lungs due to injection of a large amount of water into the gastrointestinal tract, trachea, and bronchi, which is life threatened.

Therefore, there is an urgent clinical need for a bioadhesive and biocompatible ultrasonic coupling agent that can be used in human body (including natural cavities and minimally invasive surgeries), to avoid adverse events caused by the use of water as the ultrasonic coupling agent. At the same time, a device that can deliver the ultrasonic coupling agent with a certain viscosity to the sites to be detected in the human cavities through the narrow lumen of the endoscope is also highly needed.

### SUMMARY OF THE INVENTION

In general, provided herein is an ultrasonic coupling agent for endoscopes, which can be applied to and adhered on the target sites to be detected and/or an active area of an ultrasonic detector (such as probe of the ultrasonic detector) which is able to receive and transfer the ultrasonic energy from the ultrasonic detector, such that the active area of the ultrasonic detector can easily move on the target sites to be detected, thereby effectively and safely transferring the ultrasonic energy to the target sites and/or receiving the ultrasonic energy from the target sites with very small acoustic attenuation.

In the first aspect of the present invention, provided herein is a biocompatible ultrasonic coupling agent for endoscopes, comprising a biocompatible modified starch and a pharmaceutically acceptable carrier. The biocompatible modified starch is degradable by an amylase and/or a saccharidase. The ultrasonic coupling agent produces an acoustic characteristic impedance matching the acoustic characteristic impedance of the human tissues during use for endoscopic ultrasound examination. The biocompatible modified starch is in an amount of 0.1% to 10%, or 0.1% to 9%, or 0.1% to 8%, or 0.1% to 7%, or 0.1% to 6%, or 0.1% to 5%, or 0.1% to 4%, or 0.1% to 3%, or 0.10% to 2%, or 0.10% to 1%, or 0.10% to 0.5%, or 0.10% to 0.2% of the total weight of the ultrasonic coupling agent. The pharmaceutically acceptable carrier is selected from the group consisting of normal saline, balanced salt solution, glucose solution, sterile pyrogen-free water and glycerine.

In some embodiments of the first aspect of the present invention, the ultrasonic coupling agent can produce an acoustic characteristic impedance from 1.5×10⁶ to 1.7×10⁶ Pa·s/m, during use for endoscopic ultrasound examination.

In some embodiments of the first aspect of the present invention, the biocompatible modified starch has a molecular weight from 3,000 to 2,000,000 Dalton, or 3,000 to 200,000 Dalton, or 3,000 to 100,000 Dalton, or 3,000 to 50,000 Dalton, and has a water absorbency capability from 2 to 100 times, or 5 to 75 times, or 5 to 50 times, or 2 to 10 times, or 2 to 5 times of its own weight, and has a particle size ranging from 1 to 500 μm, or 1 to 1000 μm, or 10 to 1000 μm. The biocompatible modified starch comprises at least one of pre-gelatinized starches, acid-modified starches, composite modified starches, esterified starches, etherified starches, cross-linked starches, and graft starches. The etherified starches comprise carboxymethyl starch and the salt thereof, oxidized starch and hydroxyethyl starch. The esterified starches comprise carboxymethyl starch and the salt thereof. The cross-linked starches comprise cross-linked carboxymethyl starch and the salt thereof. The pre-gelatinized starches comprise a pre-gelatinized hydroxypropyl starch diphosphate. The graft starches comprise propylene ester-carboxymethyl starch grafted copolymer and acrylic acid-carboxymethyl starch grafted copolymer. The composite modified starches comprise pre-gelatinized hydroxypropyl starch diphosphate.

In some embodiments of the first aspect of the present invention, the biocompatible modified starch may further comprise at least one of glucan, dextrin, soluble starch and water-soluble starch. The soluble starch refers to the starch lightly treated by acids or bases, and the solution thereof has good fluidity when it is heated and forms gel when it is cooled, such as α-starch, dextrin and the like.

In some embodiments of the first aspect of the present invention, the biocompatible ultrasonic coupling agent for endoscopes may further comprise one or more of pH adjusting agents, lubricants, humectants, dyes, antibacterial agents, fillers, therapeutics, preservatives, disinfectants, stabilizers, and defoamers.

In some embodiments of the first aspect of the present invention, the biocompatible ultrasonic coupling agent for endoscopes is sterilized by radiation, ozone, ethylene oxide, moist-heating and the like.

In the second aspect of the present invention, provided herein is use of a biocompatible modified starch as an ultrasonic coupling agent for endoscopes. The biocompatible modified starch comprises at least one of pre-gelatinized starches, acid-modified starches, composite modified starches, esterified starches, etherified starches, cross-linked starches, and graft starches. And the biocompatible modified starch has a molecular weight from 3,000 to 2,000,000 Dalton and has a water absorbency capability from 2 to 100 times of its own weight, and has a particle size ranging from 1 to 500 μm. The ultrasonic coupling agent produces an acoustic characteristic impedance matching the acoustic characteristic impedance of the human tissues. during its use for endoscopic ultrasound examination.

In some embodiments of the second aspect of the present invention, the biocompatible modified starch has a molecular weight from 3,000 to 200,000 Dalton, or 3,000 to 100,000 Dalton, or 3,000 to 50,000 Dalton, has a water absorbency capability from 5 to 75 times, or 5 to 50 times, or 2 to 10 times, or 2 to 5 times of its own weight, and has a particle size ranging from 1 to 1000 μm, or 10 to 1000 μm.

In some embodiments of the second aspect of the present invention, the etherified starches comprise carboxymethyl starch and the salt thereof, oxidized starch and hydroxyethyl starch. The esterified starches comprise carboxymethyl starch and the salt thereof. The cross-linked starches comprise cross-linked carboxymethyl starch and the salt thereof. The pre-gelatinized starches comprise pre-gelatinized hydroxypropyl starch diphosphate. The graft starches comprise propylene ester-carboxymethyl starch grafted copolymer and acrylic acid-carboxymethyl starch grafted copolymer. The composite modified starches comprise pre-gelatinized hydroxypropyl starch diphosphate.

In some embodiments of the second aspect of the present invention, the ultrasonic coupling agent can produce an acoustic characteristic impedance from 1.5×10⁶ to 1.7×10⁶ Pa·s/m, during use for endoscopic ultrasound examination.

In the third aspect of the present invention, provided herein is a biocompatible ultrasonic coupling agent for endoscopes, comprising an ingredient selected from the group consisting of cellulose, polyvinylpyrrolidone, polyoxyethylene, sodium alginate, glucan, hyaluronic acid, chitosan, light sensitive glue, ultrasonic sensitive glue, pH sensitive glue, gelatin and carbomer, and a pharmaceutically acceptable carrier. The ultrasonic coupling agent produces an acoustic characteristic impedance matching the acoustic characteristic impedance of the human tissues, during use for endoscopic ultrasound examination.

In some embodiments of the third aspect of the present invention, the ingredient selected from the group consisting of cellulose, polyvinylpyrrolidone, polyoxyethylene, sodium alginate, glucan, hyaluronic acid, chitosan, light sensitive glue, ultrasonic sensitive glue, pH sensitive glue, gelatin and carbomer has an amount from 0.10% to 10%, or 0.10% to 9%, or 0.10% to 8%, or 0.10% to 7%, or 0.1% to 6%, or 0.1% to 5%, or 0.1% to 4%, or 0.1% to 3%, or 0.1% to 2%, or 0.1% to 1%, or 0.1% to 0.5%, or 0.1% to 0.2% of the total weight of the ultrasonic coupling agent.

In some embodiments of the third aspect of the present invention, the ultrasonic coupling agent produces an acoustic characteristic impedance from 1.5×10⁶ to 1.7×10⁶ Pa·s/m, during use for endoscopic ultrasound examination.

In some embodiments of the third aspect of the present invention, the cellulose may be selected from the group consisting of carboxymethyl cellulose and hydroxyethyl cellulose.

In some embodiments of the third aspect of the present invention, the biocompatible ultrasonic coupling agent for endoscopes is sterilized by radiation, ozone, ethylene oxide, moist-heating and the like.

In some embodiments of the third aspect of the present invention, the biocompatible ultrasonic coupling agent for endoscopes may further comprise one or more of pH adjusting agents, lubricants, humectants, dyes, antibacterial agents, fillers, therapeutics, preservatives, disinfectants, stabilizers, and defoamers.

In the fourth aspect of the present invention, provided herein is use of an ingredient selected from the group consisting of cellulose, polyvinylpyrrolidone, polyoxyethylene, sodium alginate, glucan, hyaluronic acid, chitosan, light sensitive glue, ultrasonic sensitive glue, pH sensitive glue, gelatin and carbomer as a biocompatible ultrasonic coupling agent. The ultrasonic coupling agent produces an acoustic characteristic impedance matching the acoustic characteristic impedance of the human tissues, during use for endoscopic ultrasound examination.

In some embodiments of the fourth aspect of the present invention, the ultrasonic coupling agent produces an acoustic characteristic impedance from 1.5×10⁶ to 1.7×10⁶ Pa·s/m, during use for endoscopic ultrasound examination.

In the fifth aspect of the present invention, provided herein is a kit for endoscopic ultrasound examination, comprising the biocompatible ultrasonic coupling agent as described in the above first and third aspects, and a device for delivering the said ultrasonic coupling agent as well as a delivery tube.

The device for delivering the ultrasonic coupling agent comprises:
a hollow housing with a hollow portion for receiving the ultrasonic coupling agent to be delivered, a proximal and a distal;
a plunger disposed within the hollow portion of the housing,
a plunger rod connected to the plunger, for driving the plunger to perform reciprocating movement in the hollow portion of the housing so as to allow the ultrasonic coupling agent which is received in the hollow portion to be delivered out from the distal; and
a plunger driving mechanism having a first arm and a second arm which are pivotably connected with each other, wherein the plunger rod is driven to allow the plunger to perform reciprocating movement in the hollow portion of the housing when the first arm and the second arm pivotably rotate relative to each other.

The delivery tube is connected to the distal of the device for delivering the biocompatible ultrasonic coupling agent from the device to the site to be examined.

In some embodiments of the fifth aspect of the present invention, the first arm has a proximal and a distal. The second arm has a proximal and a distal. The distal of the first arm is connected to the proximal of the housing. The distal of the second arm is connected to the proximal of the plunger rod. When the first arm and the second arm pivotably rotate to allow the proximal of the first arm and the proximal of the second arm to move in the direction of facing each other, the second arm drives the plunger rod to allow the plunger to move to the distal within the hollow portion of the housing.

In some embodiments of the fifth aspect of the present invention, the first arm is connected to the second arm via a resilient spring, such that the first arm and the second arm can move back to the original position after the proximal of the first arm and the proximal of the second arm rotate in a direction of facing each other to drive the plunger rod to allow the plunger to move to the distal within the hollow portion of the housing.

In some embodiments of the fifth aspect of the present invention, the plunger rod is provided with thread scales thereon, which correspond to the amount of the delivered agent.

In some embodiments of the fifth aspect of the present invention, the endoscopes are selected from the group consisting of digestive tract endoscope, bronchial endoscope, urinary system endoscope, reproductive system endoscope, digestive tract ultrasound gastroscope, colonoscopy, bronchial ultrasound endoscope, urinary system ultrasound endoscope, reproductive system ultrasound endoscope, vascular ultrasound endoscope.

In some embodiments of the fifth aspect of the present invention, the kit for endoscopic ultrasound examination is sterilized by radiation, ozone, ethylene oxide, moist-heating and the like.

In the sixth aspect of the present invention, provided herein is a method for performing ultrasound examination in body cavities, including applying the biocompatible ultrasonic coupling agent for endoscopes as described in the first and the third aspects to the target sites to be examined in the cavities through the device for delivering the said ultrasonic coupling agent and the delivery tube, allowing the ultrasound probe to contact with the biocompatible ultrasonic coupling agent for endoscopes, thereby effectively and safely transferring the ultrasonic energy to the target sites and/or receiving the ultrasonic energy from the target sites with very small acoustic attenuation. The target sites in the cavities comprise the mucosal surface of the digestive tract, the mucosal surface of the respiratory tract, the mucosal surface of the genital tract or the mucosal surface of the urinary tract. The mucosa of the digestive tract includes the esophageal mucosa or the gastrointestinal mucosa. The mucosa of the respiratory tract includes the nasal mucosa, larynx mucosa, oral mucosa, trachea or bronchus mucosa. The mucosa of the urinary tract includes urethral mucosa or bladder mucosa. The mucosa of the reproductive tract includes vaginal mucosa or uterine mucosa.

In some embodiments of the sixth aspect of the present invention, the biocompatible ultrasonic coupling agent for endoscopes as described in the first and the third aspects is applied to the target sites in the cavities to be examined through the delivery device and the delivery tube as described in the above fifth aspect.

In some embodiments of the sixth aspect of the present invention, the ultrasonic coupling agent is directly delivered to the surfaces of the organs and tissues that are to be examined by the endoscopes, through the working channel (such as water channel or channel for biopsy clamp) of the endoscopes, via the delivery tube connected to the distal of the delivery device by using the above-described delivery device. Then the ultrasound probe of the endoscope directly contacts the ultrasonic coupling agent to perform ultrasonic detection and examination on the tissues and organs.

In some embodiments of the sixth aspect of the present invention, the ultrasonic coupling agent is directly delivered to the surfaces of the organs and tissues that are to be examined by the endoscopes along the outer wall of the endoscope, through the delivery tube connected to the distal of the delivery device by using the above-described delivery device. Then the ultrasound probe of the endoscope directly contacts the ultrasonic coupling agent, to perform ultrasonic detection and examination on the tissues and organs.

In some embodiments of the sixth aspect of the present invention, the ultrasonic coupling agent is delivered to a balloon connected to the distal of the endoscope along the outer wall of the endoscope, through the delivery tube connected to the distal of the delivery device by using the above-described delivery device, and then the balloon filled with the ultrasonic coupling agent is attached to the surfaces of the organs and tissues that are to be examined. Next, the ultrasound probe of the endoscope performs ultrasonic detection and examination on the tissues and organs through the balloon.

In the seventh aspect of the present invention, provided herein is a kit for preparing a biocompatible ultrasonic coupling agent for endoscopes, comprising a biocompatible modified starch and a pharmaceutically acceptable carrier. The ultrasonic coupling agent produces an acoustic characteristic impedance matching the acoustic characteristic impedance of the human tissues, during use for endoscopic ultrasound examination.

In some embodiments of the seventh aspect of the present invention, the biocompatible modified starch comprises at least one of pre-gelatinized starches, acid-modified starches, composite modified starches, esterified starches, etherified starches, cross-linked starches, and graft starches. The biocompatible modified starch has a molecular weight from 3,000 to 2,000,000 Dalton, or 3,000 to 200,000 Dalton, or 3,000 to 100,000 Dalton, or 3,000 to 50,000 Dalton, and has a water absorbency capability from 2 to 100 times, or 5 to 75 times, or 5 to 50 times, or 2 to 10 times, or 2 to 5 times of its own weight, and has a particle size ranging from 1 to 1000 μm, or 10 to 1000 μm, or 1 to 500 μm. The etherified starches comprise carboxymethyl starch and the salt thereof, oxidized starch and hydroxyethyl starch. The esterified starches comprise carboxymethyl starch and the salt thereof. The cross-linked starches comprise cross-linked carboxymethyl starch and the salt thereof. The pre-gelatinized starches comprise a pre-gelatinized hydroxypropyl starch diphosphate. The graft starches comprise propylene ester-carboxymethyl starch grafted copolymer and acrylic acid-carboxymethyl starch grafted copolymer. The composite modified starches comprise pre-gelatinized hydroxypropyl starch diphosphate.

In some embodiments of the seventh aspect of the present invention, the pharmaceutically acceptable carrier is selected from the group consisting of normal saline, balanced salt solution, glucose solution, sterile pyrogen-free water and glycerine. The kit may further comprise an instruction to indicate the ratio of the biocompatible modified starch to the pharmaceutically acceptable carrier and the preparation condition.

In some embodiments of the seventh aspect of the present invention, the ultrasonic coupling agent produces an acoustic characteristic impedance from 1.5×10⁶ to 1.7×10⁶ Pa·s/m, during use for endoscopic ultrasound examination.

In some embodiments of the seventh aspect of the present invention, the kit may further comprise anti-bacteria agents and/or therapeutic agents.

In some embodiments of the seventh aspect of the present invention, the kit for preparing a biocompatible ultrasonic coupling agent for endoscopes is sterilized by radiation, ozone, ethylene oxide, moist-heating and the like.

In the eighth aspect of the present invention, provided herein is a kit for preparing a biocompatible ultrasonic coupling agent for endoscopes, comprising an ingredient selected from the group consisting of cellulose, polyvinylpyrrolidone, polyoxyethylene, sodium alginate, glucan, hyaluronic acid, chitosan, light sensitive glue, ultrasonic sensitive glue, pH sensitive glue, gelatin and carbomer, and a pharmaceutically acceptable carrier. The ultrasonic coupling agent produces an acoustic characteristic impedance matching the acoustic characteristic impedance of the human tissues, during use for endoscopic ultrasound examination.

In some embodiments of the eighth aspect of the present invention, the pharmaceutically acceptable carrier is selected from the group consisting of normal saline, balanced salt solution, glucose solution, sterile pyrogen-free water and glycerine. The kit may further comprise an instruction to indicate the ratio of the ingredient selected from the group consisting of cellulose, polyvinylpyrrolidone, polyoxyethylene, sodium alginate, glucan, hyaluronic acid, chitosan, light sensitive glue, ultrasonic sensitive glue, pH sensitive glue, gelatin and carbomer to the pharmaceutically acceptable carrier and the preparation condition.

In some embodiments of the eighth aspect of the present invention, the kit may further comprise anti-bacteria agents and/or therapeutic agents.

In some embodiments of the eighth aspect of the present invention, the kit for preparing a biocompatible ultrasonic coupling agent for endoscopes is sterilized by radiation, ozone, ethylene oxide, moist-heating and the like.

In some embodiments of the eighth aspect of the present invention, the ultrasonic coupling agent produces an acoustic characteristic impedance from 1.5×10⁶ to 1.7×10⁶ Pa·s/m, during use for endoscopic ultrasound examination.

### BRIEF DESCRIPTION OF THE DRAWINGS

FIGS. 1A to 1K are ultrasound images obtained by using the ultrasonic coupling agent as provided herein and the control sample.

FIG. 2 is a schematic diagram to illustrate the use of the kit for endoscopic ultrasound examination according to one embodiment as described herein.

FIG. 3 is a schematic diagram to illustrate the use of the kit for endoscopic ultrasound examination according to one embodiment as described herein.

FIG. 4 is a schematic diagram to illustrate the use of the kit for endoscopic ultrasound examination according to one embodiment as described herein.

FIG. 5 is a schematic diagram to illustrate the use of the kit for endoscopic ultrasound examination according to one embodiment as described herein.

### DETAILED DESCRIPTION OF THE INVENTION

The terminology used herein is for the purpose of describing particular embodiments only and is not intended to be any limitation of the invention. As used herein, the singular forms “a”, “an” and “the” are intended to include the plural forms as well, unless the context clearly indicates otherwise.

### Definition

The term “biocompatibility” or “biocompatible” as used herein refers to ability of tissues in a living body to perform an appropriate response to an inactive material. Generally, it refers to the compatibility of the materials with the host. Evaluation on biocompatibility mainly follows biosafety principles, i.e., elimination of injurious effect of biological materials on human tissues and organs, such as allergenicity, cytotoxicity and carcinogenicity. In addition, according to the sites on which the biological materials are to be applied, after the biological materials are directly used on the tissues and organs in the human body, they are required to be degradable and/or absorbable by organisms and tissues. Since the biocompatible ultrasonic coupling agent as described herein can be used for endoscopic ultrasound examination in human cavities, the biocompatibility as used herein especially refers to the absorbability and non-allergenicity of the materials in full compliance with biosafety principles.

The term “absorbable/degradable or degraded” as used herein means that a substance can be gradually destroyed in the organism (chemical hydrolysis, enzymatic hydrolysis, or phagocytic action, etc.), including morphological and structural destruction and performance changes, and the resultant products after degradation can be absorbed and metabolized by the organism, or can be self-decomposed. During absorbency or degradation, no by-products harmful to the human body is produced.

The term “water absorbency capability” as used herein refers to the ratio of the mass or volume of water absorbed by unit mass or volume of the water absorbent to the volume or mass of the water absorbent.

The term “pharmaceutically acceptable carrier” as used herein means that the carrier does not produce any toxic or adverse side effects after applying to a human, and is compatible with the active ingredients dissolved and/or suspended and/or complexed and/or mixed therein. The term “pharmaceutically acceptable carrier” includes any and all solvents, dispersion media, isotonic agents, excipients, and the like, which are known to those of ordinary skill in the art, and combinations thereof.

For the operator who operates the device for delivering the ultrasonic coupling agent as described herein, the “proximal end” used herein refers to the portion that is closest to the operator.

For the operator who operates the device for delivering the ultrasonic coupling agent as described herein, the “distal end” used herein refers to the portion that is farthest away from the operator.

The ultrasonic coupling agent for endoscopes as provided herein has the following advantages:
1) It meets the basic performance requirements for the common ultrasonic coupling agent, with reduced loss of ultrasonic energy and high ultrasonic image definition.
2) It has bioadhersivity, and thus can be adhered to the mucosa of the cavities and organs for a period time that is sufficient to perform endoscopic ultrasound examination and treatment, and it is able to stretch the wrinkles of the natural cavity of the human body.
3) It is non-toxic, and can be used in vivo, with good biocompatibility and absorbency (can be fully absorbed/degraded in the human body).
4) It will not block the gastrointestinal tract, pancreaticobiliary duct, urethra, ureter and other natural human cavities.
5) It can be easily injected into the organ cavities in the body through the working channel of the endoscope (such as the biopsy clamp channel).
6) It does not corrode or damage the ultrasound probe and endoscope.
7) It has acid and alkali resistance.
8) It can be easily cleaned without blocking the working channel of the endoscope.
9) It can be easily sterilized to meet sterile requirements.
10) It has reasonable cost and can be easily obtained.

The aspects of the present invention will be described in greater detail by referring to the working examples as below. The working examples are illustrative without making limitation to the scope and the spirit of the present invention.

### Example 1. Biocompatible Ultrasonic Coupling Agent for Endoscopes

Table 1 lists the starting materials #1 to #10, which are dispersed into normal saline (NS) at varied amounts to prepare ultrasonic coupling agent samples #11 to #20. The chemical and physical parameters of the starting materials #1 to #10 are listed in Table 1. The amounts of the starting materials and NS that are used to prepare the ultrasonic coupling agent samples #11 to #20 and the performance parameters of the resultant ultrasonic coupling agent samples #11 to #20 are listed in Table 2.

TABLE 1

| | | Molecular | Particle | | Water | Viscosity |
|---|---|---|---|---|---|---|
| No. | Starting Material | Weight | Size | | Absorbancy | (mPa · s) |
| #1 | Polyoxyethylene | 100,000-10,000,000 | 0.5~2000 | μm | 21 | 30-1000 in 1% |
| | (PEO) | | | | | aqueous at 37° C. |
| #2 | Polyvinylpyrrolidone | 8,000-1,500,000 | 5~1000 | μm | 9 | 50-1000 in 5% |
| | (PVP) | | | | | aqueous at 37° C. |
| #3 | hydroxyethylcellulose | 30,000-500,000 | 10~1000 | μm | 20 | 5-60,000 in 2% |
| | (HEC) | | | | | aqueous at 20° C. |
| #4 | carboxymethylcellulose | 5,000-20,000 | 100~1000 | μm | 29 | 1000-50000 in 1% |
| | (CMC) | | | | | aqueous at 37° C. |
| #5 | Arabic gum | 200,000-1,000,000 | 5~1000 | μm | 10 | 50-1000 in 1% |
| | | | | | | aqueous at 37° C. |
| #6 | tragacanth | 100,000-1,000,000 | 100~1500 | μm | 15 | 400-600 in 1% |
| | | | | | | aqueous at 25° C. |
| #7 | Carbomer | 1,000,000-5,000,000 | 10~1000 | μm | 19 | 1000-50,000 in 0.5% |
| | | | | | | aqueous at 37° C. |
| #8 | sodium alginate | 30,000-250,000 | 100~1500 | μm | 23 | 1000-50000 in 1% |
| | | | | | | aqueous at 37° C. |
| #9 | hydroxyethyl starch | 100,000-300,000 | 10~1000 | μm | 4 | 1-100 in 1% |
| | (HES) | | | | | aqueous at 37° C. |
| #10 | glucan | 5,000-2,000,000 | 10~1000 | μm | 5 | 1-100 in 2% |
| | | | | | | aqueous at 37° C. |

TABLE 2

| | | | | | acoustic | slope of sound | | |
|---|---|---|---|---|---|---|---|---|
| | | | | sound | characteristic | attenuation | | |
| | | | | velocity | impedance | coefficient | | |
| | | | | (35° C.) | (35° C.) | (35° C.) | Viscosity | Adhesion |
| No. | Formula | Appearance | pH | m/s | Pa · s/m | dB/(cm · MHz) | (mPa · s) | (gf) |
| Standard | | Colorless or light- | 5.5~8.0 | 1520~1620 | 1.5 × 10⁶~1.7 × 10⁶ | ≤0.05 | / | / |
| Requirements | | colored transparent gel, | | | | | | |
| | | no or only a few | | | | | | |
| | | bubbles, no insoluble | | | | | | |
| | | foreign matter | | | | | | |
| #11 | Sample #1 + NS, | Good | 8.0 | 1520~1620 | 1.5 × 10⁶~1.7 × 10⁶ | ≤0.05 | 783 | 16.0 |
| | with 1 wt % | | | | | | | |
| | sample #1 | | | | | | | |
| #12 | Sample #2 + NS, | Good | 6.2 | 1520~1620 | 1.5 × 10⁶~1.7 × 10⁶ | ≤0.05 | 350 | 10.6 |
| | with 5 wt % | | | | | | | |
| | sample #2 | | | | | | | |
| #13 | Sample #3 + NS, | Good | 6.5 | 1520~1620 | 1.5 × 10⁶~1.7 × 10⁶ | ≤0.05 | 29645 | 19.8 |
| | with 1 wt % | | | | | | | |
| | sample #3 | | | | | | | |
| #14 | Sample #4 + NS, | Good | 7.8 | 1520~1620 | 1.5 × 10⁶~1.7 × 10⁶ | ≤0.05 | 23680 | 19.5 |
| | with 0.5 wt % | | | | | | | |
| | sample #4 | | | | | | | |
| #15 | Sample #5 + NS, | Good | 7.0 | 1520~1620 | 1.5 × 10⁶~1.7 × 10⁶ | ≤0.05 | 296 | 6.3 |
| | with 2 wt % | | | | | | | |
| | sample #5 | | | | | | | |
| #16 | Sample #6 + NS, | Good | 5.5 | 1520~1620 | 1.5 × 10⁶~1.7 × 10⁶ | ≤0.05 | 514 | 11.2 |
| | with 1 wt % | | | | | | | |
| | sample #6 | | | | | | | |
| #17 | Sample #7 + NS, | Good | 8.0 | 1520~1620 | 1.5 × 10⁶~1.7 × 10⁶ | ≤0.05 | 25440 | 20.5 |
| | with 0.2 wt % | | | | | | | |
| | sample #7 | | | | | | | |
| #18 | Sample #8 + NS, | Good | 6.4 | 1520~1620 | 1.5 × 10⁶~1.7 × 10⁶ | ≤0.05 | 21835 | 19.8 |
| | with 0.5 wt % | | | | | | | |
| | sample #8 | | | | | | | |
| #19 | Sample #9 + NS, | Good | 7.0 | 1520~1620 | 1.5 × 10⁶~1.7 × 10⁶ | ≤0.05 | 43 | 4.2 |
| | with 6 wt % | | | | | | | |
| | sample #9 | | | | | | | |
| #20 | Sample #10 + NS, | Good | 7.0 | 1520~1620 | 1.5 × 10⁶~1.7 × 10⁶ | ≤0.05 | 165 | 6.4 |
| | with 5 wt % | | | | | | | |
| | sample #10 | | | | | | | |

The performance parameters listed in Table 2 were measured according to the methods specified in the industry standard for medical ultrasonic coupling agents (YY0299). From Table 2, it can be seen that the ultrasonic coupling agent of the present invention can achieve a sound velocity from 1520 to 1620 m/s, with the slope of the sound attenuation coefficient ≤0.05 dB/(cm·MHz), and an acoustic characteristic impedance ranging from 1.5×10⁶ to 1.7×10⁶ Pa·s/m. It can be seen that the ultrasonic coupling agent of the present invention produces an acoustic characteristic impedance that perfectly matches the acoustic characteristic impedance of the tissues in human cavities, and has low sound attenuation, suitable for use as a coupling agent for endoscopic ultrasound examination.

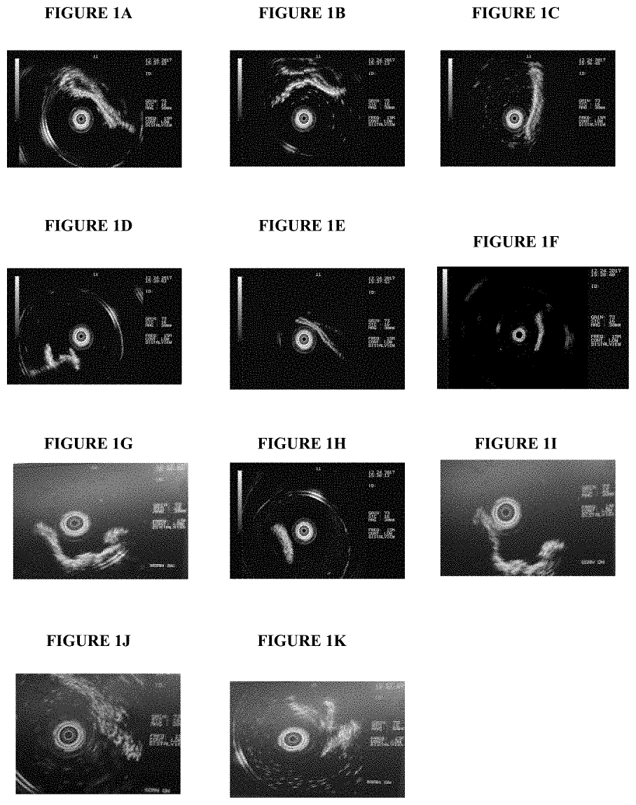

The above samples #11 to #20 are placed in a 50 mL centrifuge tube. A gauze with an area of 1.5×1.5 cm is dipped into each sample. Then the ultrasonic probe is inserted into each sample and the image detected by the ultrasonic probe is recorded. The control sample is normal saline. FIGS. 1A to 1K show the images detected by the ultrasonic probe in the samples #11 to #20, in which FIG. 1K is the result from the control sample.

From FIGS. 1A to 1K, it can be seen that the images returned by the ultrasonic coupling agent of the present invention have high definition and less white noise. Therefore, the ultrasonic coupling agent of the present invention is suitable for use as a coupling agent for endoscopic ultrasound examination.

### Example 2. Use of the Biocompatible Ultrasonic Coupling Agent in In Vivo Examination

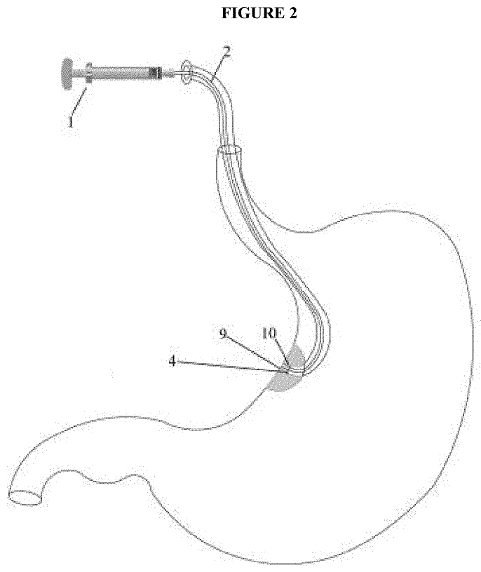

FIG. 2 is a schematic diagram to illustrate the use of the kit for endoscopic ultrasound examination according to one embodiment as described herein. As shown in FIG. 2, the ultrasonic coupling agent prepared according to Example 1 is directly delivered to the surface 9 of the gastric mucosa that is to be examined by the endoscope, through the working channel (such as water channel or channel for biopsy clamp) of the endoscope, via the delivery tube 2 connected to the distal of the delivery device 1 according to the present invention. Then the ultrasound probe 4 of the endoscope directly contacts the ultrasonic coupling agent 10, to perform ultrasonic detection and examination on the tissues and organs.

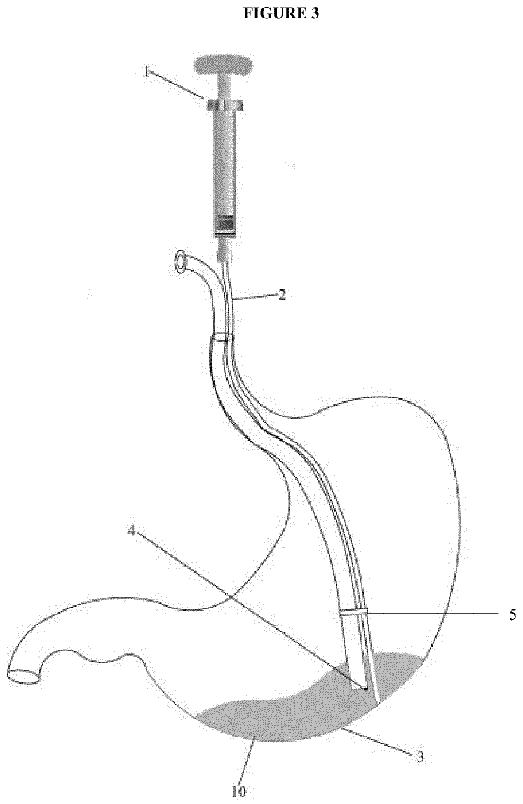

FIG. 3 is a schematic diagram to illustrate the use of the kit for endoscopic ultrasound examination according to one embodiment as described herein. As shown in FIG. 3, the ultrasonic coupling agent prepared according to Example 1 is directly delivered to lesion site in the stomach which is to be examined by the endoscope along the outer wall of the endoscope (with the delivery tube 2 fixed along the outer wall of the endoscope by a fastener 5), through the delivery tube 2 connected to the distal of the delivery device 1 according to the present invention. Then the ultrasound probe 4 of the endoscope directly contacts the ultrasonic coupling agent 10, to perform ultrasonic detection and examination on the tissues and organs.

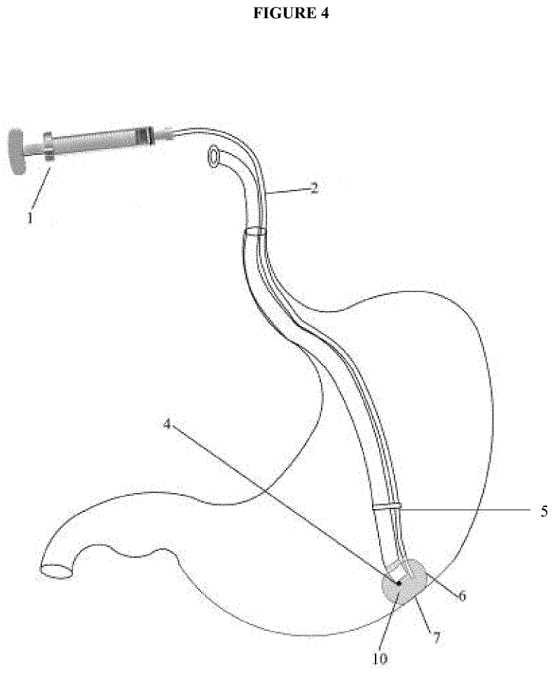

FIG. 4 is a schematic diagram to illustrate the use of the kit for endoscopic ultrasound examination according to one embodiment as described herein. As shown in FIG. 4, the ultrasonic coupling agent prepared according to Example 1 is delivered to a balloon 6 connected to the distal of the endoscope along the outer wall of the endoscope (with the delivery tube 2 fixed along the outer wall of the endoscope by a fastener 5), through the delivery tube 2 connected to the distal of the delivery device 1 according to the present invention, and then the balloon 6 filled with the ultrasonic coupling agent 10 is attached to the lesion site 7 in the stomach which is to be examined. Next, the ultrasound probe 4 of the endoscope performs ultrasonic detection and examination on the tissues and organs through the balloon.

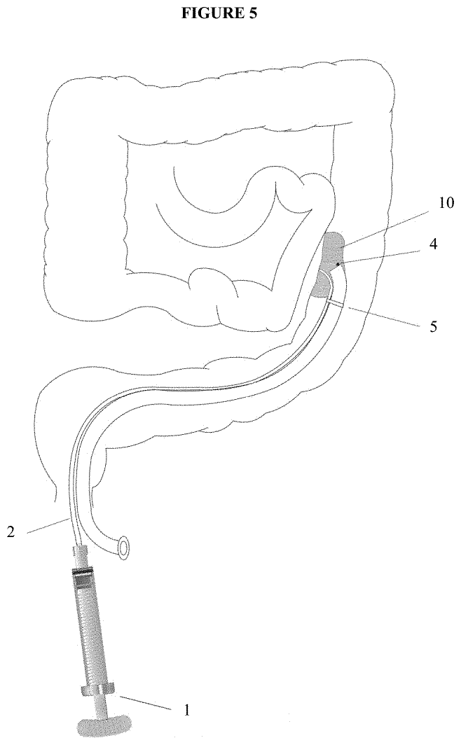

FIG. 5 is a schematic diagram to illustrate the use of the kit for endoscopic ultrasound examination according to one embodiment as described herein. As shown in FIG. 5, the ultrasonic coupling agent prepared according to Example 1 is directly delivered to lesion site in the intestine which is to be examined by the endoscope along the outer wall of the endoscope (with the delivery tube 2 fixed along the outer wall of the endoscope by a fastener 5), through the delivery tube 2 connected to the distal of the delivery device 1 according to the present invention. Then the ultrasound probe 4 of the endoscope directly contacts the ultrasonic coupling agent 10, to perform ultrasonic detection and examination on the tissues and organs.

### Example 3. Effect of the Biocompatible Ultrasonic Coupling Agent in Endoscopic Ultrasound Examination In Vivo

This example illustrates the effect of the ultrasonic coupling agent samples #11 to #20 prepared according to Example 1 in gastroscopy ultrasound examination on Bama miniature pig.

1. Ultrasonic coupling agent: samples #11 to #20 prepared according to Example 1

2. Animals: Bama miniature pig, with body weight of 40 kg

3. Experimental method: After general anesthesia, the Bama miniature pig lied on its back on the operating table with its limbs fixed. The Olympus GIF-XQ240 electronic gastroscope was used to enter from the mouth. 2 ml of normal saline was submucosally injected into the pig's esophagus to form submucosal bulges. Then the biocompatible ultrasonic coupling agents prepared according to Example 1 were applied on the bulging section through the working channel of the gastroscope via the delivery tube by using the delivery device. The control group was continuously perfused with NS. The P2615-M Fuji endoscopic ultrasound probe was used to perform detection. The ultrasonic images and the definition thereof were recorded and compared.

### Example 4. Effect of the Biocompatible Ultrasonic Coupling Agent in Endoscopic Ultrasound Examination In Vivo

This example illustrates the effect of the ultrasonic coupling agent samples #11 to #20 prepared according to Example 1 in gastroscopy ultrasound examination on Bama miniature pig.

1. Ultrasonic coupling agent: samples #11 to #20 prepared according to Example 1

2. Animals: Bama miniature pig, with body weight of 40 kg

3. Experimental method: After general anesthesia, the Bama miniature pig lied on its back on the operating table with its limbs fixed. The Olympus ultrasonic endoscope was used to enter from the mouth. 2 ml of normal saline was submucosally injected into the anterior wall of the upper third of the pig's stomach to form submucosal bulges. Then the biocompatible ultrasonic coupling agents prepared according to Example 1 were applied on the bulging section through the working channel of the gastroscope via the delivery tube by using the delivery device. The control group was continuously perfused with NS. The ultrasound probe was used to perform detection. The ultrasonic images and the definition thereof were recorded and compared.

1. (canceled)

2. (canceled)

3. (canceled)

4. (canceled)

5. (canceled)

6. (canceled)

7. (canceled)

8. (canceled)

9. (canceled)

10. A method of using an ultrasonic coupling agent during an endoscopic ultrasound examination, comprising:
acquiring the ultrasonic coupling agent wherein the ultrasonic coupling agent comprises a biocompatible modified starch, wherein the biocompatible modified starch comprises at least one of pre-gelatinized starches, acid-modified starches, composite modified starches, esterified starches, etherified starches, cross-linked starches, or graft starches and wherein the biocompatible modified starch has a molecular weight from 3,000 to 2,000,000 Dalton, has a water absorbency capability from 2 to 100 times of its own weight, and has a particle size ranging from 1 to 1000 μm; and
applying the ultrasonic coupling agent proximate human tissue during the endoscopic ultrasound examination, wherein the biocompatible modified starch is adapted to produces an acoustic characteristic impedance matching an acoustic characteristic impedance of the human tissues.

11. The method of claim 10, wherein the biocompatible modified starch has a molecular weight in a range of 3,000 to 200,000 Dalton, or in a range of 3,000 to 100,000 Dalton, or in a range of 3,000 to 50,000 Dalton.

12. The method of claim 10, wherein the biocompatible modified starch has a water absorbency capability in a range of 5 to 75 times, in a range of 5 to 50 times, in a range of 2 to 10 times, or in a range of 2 to 5 times of its own weight.

13. The method of claim 10, wherein the biocompatible modified starch has a particle size ranging 1 to 500 μm, or in a range of 10 to 1000 μm.

14. The method of claim 10, wherein:
the etherified starches comprise at least one of a carboxymethyl starch and a salt thereof, an oxidized starch or a hydroxyethyl starch;
the esterified starches comprise carboxymethyl starch and a salt thereof;
the cross-linked starches comprise cross-linked carboxymethyl starch and a salt thereof,
the pre-gelatinized starches comprise a pre-gelatinized hydroxypropyl starch diphosphate;
the graft starches comprise propylene ester-carboxymethyl starch grafted copolymer and acrylic acid-carboxymethyl starch grafted copolymer; and
the composite modified starches comprise pre-gelatinized hydroxypropyl starch diphosphate.

15. The use-method of claim 10, wherein the acoustic characteristic impedance is in a range of 1.5×10⁶ to 1.7×10⁶ Pa·s/m.

16. A biocompatible ultrasonic coupling agent adapted to be used in an endoscopic procedure, comprising:
a composition selected from the group consisting of cellulose, polyvinylpyrrolidone, polyoxyethylene, sodium alginate, glucan, hyaluronic acid, chitosan, light sensitive glue, ultrasonic sensitive glue, pH sensitive glue, gelatin and carbomer; and
a pharmaceutically acceptable carrier, wherein the ultrasonic coupling agent is adapted to produces an acoustic characteristic impedance matching an acoustic characteristic impedance of the human tissue during the endoscopic procedure.

17. The biocompatible ultrasonic coupling agent of claim 16, wherein the composition is selected from the group consisting of cellulose, polyvinylpyrrolidone, polyoxyethylene, sodium alginate, glucan, hyaluronic acid, chitosan, light sensitive glue, ultrasonic sensitive glue, pH sensitive glue, gelatin and carbomer and wherein the composition is in an amount from 0.1% to 10%, or 0.1% to 9%, or 0.1% to 8%, or 0.1% to 7%, or 0.1% to 6%, or 0.1% to 5%, or 0.1% to 4%, or 0.1% to 3%, or 0.1% to 2%, or 0.1% to 1%, or 0.1% to 0.5%, or 0.1% to 0.2% of a total weight of the ultrasonic coupling agent.

18. The biocompatible ultrasonic coupling agent of claim 16, wherein the ultrasonic coupling agent is adapted to produces an acoustic characteristic impedance from 1.5×10⁶ to 1.7×10⁶ Pa·s/m.

19. (canceled)

20. (canceled)

21. (canceled)

22. (canceled)

23. The method of claim 10, wherein the biocompatible modified starch is degradable by an amylase and/or a saccharidase.

24. The method of claim 10, wherein the biocompatible modified starch is in an amount of 0.1% to 10%, or 0.1% to 9%, or 0.1% to 8%, or 0.1% to 7%, or 0.1% to 6%, or 0.1% to 5%, or 0.1% to 4%, or 0.1% to 3%, or 0.1% to 2%, or 0.1% to 1%, or 0.1% to 0.5%, or 0.1% to 0.2% of a total weight of the ultrasonic coupling agent.

25. The method of claim 10, wherein the ultrasonic coupling agent comprises a pharmaceutically acceptable carrier and wherein the pharmaceutically acceptable carrier is selected from the group consisting of normal saline, balanced salt solution, glucose solution, sterile pyrogen-free water and glycerine.

26. The method of claim 10, wherein the biocompatible modified starch has a water absorbency capability in a range of 5 to 75 times, in a range of 5 to 50 times, in a range of 2 to 10 times, or in a range of 2 to 5 times of its own weight.

27. The method of claim 10, wherein the biocompatible modified starch has a particle size ranging from 1 to 500 μm or 10 to 1000 μm.

28. A biocompatible ultrasonic coupling agent adapted to be used in an endoscopic procedure, comprising:
a biocompatible modified starch, wherein the biocompatible modified starch comprises at least one of pre-gelatinized starches, acid-modified starches, composite modified starches, esterified starches, etherified starches, cross-linked starches, or graft starches and wherein the biocompatible modified starch has a molecular weight from 3,000 to 2,000,000 Dalton, has a water absorbency capability from 2 to 100 times of its own weight, and has a particle size ranging from 1 to 1000 μm; and
a pharmaceutically acceptable carrier, wherein the ultrasonic coupling agent is adapted to produce an acoustic characteristic impedance matching an acoustic characteristic impedance of human tissue during the endoscopic procedure.

29. The biocompatible ultrasonic coupling agent of claim 28, wherein the biocompatible modified starch has a molecular weight in a range of 3,000 to 200,000 Dalton, or in a range of 3,000 to 100,000 Dalton, or in a range of 3,000 to 50,000 Dalton.

30. The biocompatible ultrasonic coupling agent of claim 28, wherein the biocompatible modified starch has a water absorbency capability in a range of 5 to 75 times, in a range of 5 to 50 times, in a range of 2 to 10 times, or in a range of 2 to 5 times of its own weight.

31. The biocompatible ultrasonic coupling agent of claim 28, wherein the acoustic characteristic impedance is in a range of 1.5×10⁶ to 1.7×10⁶ Pa·s/m.

32. The biocompatible ultrasonic coupling agent of claim 28, wherein the biocompatible modified starch is degradable by an amylase and/or a saccharidase.

33. The biocompatible ultrasonic coupling agent of claim 28, wherein the biocompatible modified starch is in an amount of 0.1% to 10%, or 0.1% to 9%, or 0.1% to 8%, or 0.1% to 7%, or 0.1% to 6%, or 0.1% to 5%, or 0.1% to 4%, or 0.1% to 3%, or 0.1% to 2%, or 0.1% to 1%, or 0.1% to 0.5%, or 0.1% to 0.2% of a total weight of the ultrasonic coupling agent.
